(19) United States
(12) Patent Application Publication
(10) Pub. No.: US 2014/0275995 A1
(43) Pub. Date: Sep. 18, 2014

(54) DEFINED BORDERS

(71) Applicant: VOLCANO CORPORATION, San Diego, CA (US)

(72) Inventor: David Sheehan, Poway, CA (US)

(73) Assignee: VOLCANO CORPORATION, San Diego, CA (US)

(21) Appl. No.: 14/202,300

(22) Filed: Mar. 10, 2014

Related U.S. Application Data
(60) Provisional application No. 61/776,858, filed on Mar. 12, 2013.

(51) Int. Cl. A61B 5/00

(52) U.S. Cl. CPC A61B 5/0035
USPC 600/424

(57) ABSTRACT

The present invention relates to use of co-registered data to virtually recreate a section of a vessel in an external image, in which the section of the vessel cannot be imaged using an external imaging modality. In certain aspects, a method of the invention includes obtaining external imaging data of a vessel, wherein data representing a specific portion of the vessel is absent from the external imaging data, obtaining intraluminal imaging data of the specific portion of the vessel, and co-registering the external imaging data with the intraluminal imaging data to construct an external image of the vessel that includes the specific portion of the vessel.

### RELATED APPLICATION

The present application claims the benefit of and priority to U.S. Provisional No. 61/776,858, filed Mar. 12, 2013, which is incorporated by reference in its entirety.

### TECHNICAL FIELD

The invention generally relates to imaging a vessel that uses external and internal imaging modalities.

### BACKGROUND

Cardiovascular disease frequently arises from the accumulation of atheromatous deposits on inner walls of vascular lumen, particularly the arterial lumen of the coronary and other vasculature, resulting in a condition known as atherosclerosis. These deposits can have widely varying properties, with some deposits being relatively soft and others being fibrous and/or calcified. In the latter case, the deposits are frequently referred to as plaque. These deposits can restrict blood flow, which in severe cases can lead to myocardial infarction.

Conventional cardiovascular imaging includes the use of external imaging methods, such as x-ray angiography to image a vessel from the outside. Angiography is a medical imaging technique used to visualize a lumen of blood vessels and organs of the body, with particular interest in the arteries, veins and the heart chambers. This is traditionally done by injecting a radio-opaque flow contrast agent into the blood vessel and imaging with X-ray based techniques such as fluoroscopy.

Chronic total occlusions and other constricted vessels (such as those associated with Coronary microvasculature disease) pose significant problems to imaging blood vessels by x-ray angiography. The flow contrast used to visual the vessel is often blocked by the chronic total occlusion, preventing imaging of the occluded vessel segment. The resulting angiogram includes an image of a vessel prior to the chronic total occlusion, but does not include a discernible image or any image of the occluded vessel segment or portions of the vessel distal to the occlusion. In certain instances, the chronic total occlusion is shown as an undeterminable vessel break or gap on an angiogram. For example, the body may compensate for an occlusion in a primary vessel by having peripheral vessels (proximal to the occlusion) bypass the chronic total occlusion and reconnect to the primary vessel (distal to the occlusion) in order to maintain blood flow in the primary vessel. In those instances, flow contrast is able to reach the vessel before and after the chronic total occlusions. The resulting angiogram shows two image-able portions of the vessel separated by a gap where nothing is determinable.

The failure to clearly image the vessel segment containing the chronic total occlusion poses several challenges during a concurrent or subsequent percutaneous coronary invention procedure. The manipulation of interventional catheters through the chronic total chronic occlusion during the procedure without the ability to identify the occluded vessel's luminal boundaries involves risk of arterial dissection, perforation, and cardiac tamponade. In addition, the blind navigation through the chronic total occlusion increases procedural times, which increases risk of side effects and injury associated with prolonged exposure to the flow contrast and x-rays.

### SUMMARY

The invention uses a combination of internal and external imaging data sets to provide an external image of the vasculature that includes a vessel segment which is not present in an image obtained by an external imaging device alone. Methods and systems of the invention obtain intraluminal imaging data within and surrounding a chronic total occlusion in a vessel. The obtained intraluminal imaging data is then co-registered with external imaging data in order to provide visualization of the occluded vessel segment in a vasculature map image (e.g. external image of the vasculature). This advantageously allows one to assess the severity and degree of the chronic total occlusion in the vessel prior to a coronary invention procedure and reduces complications associated with blind navigation of the chronic total occlusion.

Methods of the invention are accomplished by obtaining external imaging data of a vessel, in which data representing a specific portion of the vessel is absent from the external imaging data, and obtaining intraluminal imaging data of that specific portion of the vessel. The external imaging data is co-registered with the intraluminal imaging data in order to construct an external image of the vessel that includes the specific portion of the vessel. The intraluminal imaging data is used to fill in the data representing the specific vessel portion, which is missing from the external data set, in order to produce a more complete external image of the vessel. As a result, systems and methods of the invention are able to fill in data gaps observed when using an external imaging modality alone.

The specific portion of the vessel absent from the external imaging data is typically a vessel segment having a chronic total occlusion or other constriction of blood flow. However, any vessel segment that is unable to be imaged with an external imaging device alone may be imaged or reconstructed using methods of the invention.

The intraluminal imaging data may be obtained by using any known intraluminal imaging technique, such as intravascular ultrasound or optical coherence tomography techniques. In certain embodiments, the intraluminal imaging data is obtained by inserting an intraluminal device for imaging into the chronic total occlusion, and imaging the total chronic occlusion to obtain the intraluminal imaging data. The intraluminal imaging device may be a catheter or a guidewire. In certain embodiments, the intraluminal device is an imaging guidewire, which, due to its size, is easier to insert into one or more micro-channels (e.g. endothelialized micro-channels that transverse the occlusion) of the chronic total occlusion. The external imaging data may be obtained using any known external imaging technique including, for example, angiography/fluoroscopy, computed tomography, magnetic resonance imaging, and ultrasound imaging.

### BRIEF DESCRIPTION OF THE DRAWINGS

FIG. 1A depicts an angiogram of a vessel with a total chronic occlusion obtained with an angiography imaging modality.

FIG. 1B shows a certain portion of the primary vessel that is absent from the angiogram (as indicated by the dashed lined) depicted in FIG. 1A.

FIG. 2A depicts another angiogram of a vessel with a chronic occlusion obtained with an angiography imaging modality.

FIG. 2B shows a certain portion of the primary vessel that is absent from the angiogram (as indicated by the dashed lined) depicted in FIG. 2A.

FIG. 3 is a graphical illustration of a three dimensional length of artery, including a highly diseased segment.

FIG. 4 is a graphical illustration of a portion of the artery depicted in FIG. 3 with a longitudinal section removed along lines 2 to illustratively depict different elements of atherosclerotic plaque.

FIG. 5A is a graphical illustration of the artery from FIGS. 3 and 4 wherein an imaging catheter has been inserted in the artery.

FIG. 5B is detailed view of a section of the artery depicted in FIG. 5A including an imaging catheter in the artery.

FIGS. 6A-6C depict an imaging catheter being inserted into and past a chronic total occlusion in order to provide imaging data for a vessel section that is absent from or not discernible in an external image.

FIG. 7 is a flowchart depicting a set of exemplary steps for creating a co-registered three-dimensional map of the vasculature using external and intraluminal imaging data.

FIG. 8 illustratively depicts a vessel reconstruction graphical image based on image creation techniques.

FIGS. 9A-9B illustratively depicts a vessel reconstruction of an external image co-registered with intraluminal imaging data, using intraluminal imaging data to obtain the missing image data due to a chronic total occlusion.

FIGS. 10A-10B illustratively depicts another vessel reconstruction of an external image co-registered with intraluminal imaging data, using intraluminal imaging data to obtain the missing image data due to a chronic total occlusion.

FIG. 11 illustratively depicts a graph including two separate sequences of values corresponding to lumen area, in relation to image frame number and linear displacement along an imaged vessel, prior to axial registration adjustment.

FIG. 12 illustratively depicts a graph including two separate sequences of values corresponding to lumen area, in relation to image frame number and linear displacement along an imaged vessel, after axial registration adjustment.

FIG. 13 illustratively depicts a graphical display of angiography and vessel (e.g., IVUS) images on a single graphical display prior to axial registration adjustment.

FIG. 14 illustratively depicts a graphical display of angiography and vessel (e.g., IVUS) images superimposed on a single graphical display after axial registration adjustment.

FIG. 15 illustratively depicts the process of circumferential registration of angiography and vessel (e.g., IVUS) image sets.

FIG. 16 illustratively depicts angular image displacement in relation to circumferential registration of angiography and vessel (e.g., IVUS) image sets; and

FIG. 17 illustratively depicts a graph of actual and best fit rotational angle corrections displayed in relation to image frame number.

FIG. 18 is block diagram of a system of the invention for co-registering intraluminal imaging data with external imaging data.

FIG. 19 is a block diagram of a networked system for co-registering intraluminal imaging data with external imaging data.

### DETAILED DESCRIPTION

The invention generally relates to composite co-registered images generated by a first graphical image rendered from a first type of imaging data and a second graphical image rendered from a second type of data. Typically, the co-registered images are formed using intraluminal data sets and external imaging data sets (e.g. images of the vasculature taken from outside the body). In particular aspects, systems and methods of the invention utilize intraluminal image data to fill in gaps observed in external images of the vasculature due to the presence of a chronic total occlusion or a constricted vessel.

According to certain aspects, systems and methods of the invention involve obtaining external imaging data of a vessel, in which data representing a specific portion is absent from the external imaging data, obtaining intraluminal imaging data of the specific portion of the vessel, and co-registering the external imaging data with the intraluminal imaging data to construct an external image of the vessel that includes the specific portion of the vessel.

These aspects of the invention are accomplished by taking an image of a vessel with an external imaging modality, taking an intraluminal image of the vessel with an intraluminal imaging device, and co-registering the external image with the intraluminal image. Indiscernible vessel segments present within the external image can be filled in with the co-registered intraluminal image in order to provide a more complete external image of the vessel.

In certain embodiments, an indiscernible vessel segment is a segment of a vessel with a chronic total occlusion or other constriction that prevents the occluded segment from being imaged by an external imaging modality (e.g. blocks flow contrast media required for radiological imaging). In some instances, an indiscernible vessel segment includes portion of the vessel beyond the occluded section, which also lacks flow contrast required for imaging due the occlusion. In some instances, the indiscernible vessel segment in an image is shown as a gap between two otherwise image-able sections of the vessel. For example, a body of a subject may compensate for a total chronic occlusion in a primary vessel by having peripheral vessels bypass a total chronic occlusion in the primary vessel to maintain blood flow. In this situation, an external image of the vasculature would show two image-able portions of a vessel separated by a gap where nothing is discernible.

FIG. 1A depicts an angiogram of a vessel 500 with a total chronic occlusion obtained with an angiography imaging device. As shown in FIG. 1A, a certain portion of primary vessel 500 extending beyond line 502 is not present in the angiogram due to a total occlusion present in the primary vessel 500 around line 502. FIG. 1B shows the certain portion 510 of the primary vessel 500 absent from the angiogram (as indicated by the dashed lined) depicted in FIG. 1A. In order to generate an angiogram showing the certain portion 510 of the vessel 500, methods of the invention provide for obtaining intraluminal imaging data of the certain portion 510 with an imaging catheter or guidewire and co-registering the intraluminal imaging data with the angiogram.

FIG. 2A depicts another angiogram of a vessel 512 with a chronic occlusion obtained with an angiography imaging modality. As shown in FIG. 2A, a certain portion 504 of the vessel 512 is absent from the angiogram due to a chronic total occlusion. In this instance, however, the primary vessel 512 distal to the chronic total occlusion is present in the angiogram. This is because the vasculature has a peripheral vessel 506 bypassing the total occlusion in order to maintain some blood flow through primary vessel 512. FIG. 2B shows the certain portion 504 of the primary vessel 512 absent from the angiogram (as indicated by the dashed lined) depicted in FIG. 2A. In order to generate an angiogram showing the certain portion 504 of the vessel 512, methods of the invention provide for obtaining intraluminal imaging data of the certain portion 504 with an imaging catheter or guidewire and co-registering the intraluminal imaging data with the angiogram.

The following describes methods of obtaining intraluminal imaging data and methods of co-registering the intraluminal imaging data with external imaging data, such as an angiogram.

In FIGS. 3 and 4, a diseased artery 5 with a lumen 10 is shown. Blood flows through the artery 5 in a direction indicated by arrow 15 from proximal end 25 to distal end 30. A stenotic area 20 is seen in the artery 5. FIG. 4 shows a sectioned portion of the stenotic area 20 of the artery 5. An artery wall 35 consists of three layers, an intima 40, a media 45 and an adventitia 55. An external elastic lamina (EEL) 50 is the division between the media 45 and the adventitia 55. A stenosis 60 is located in the artery 5 and limits blood flow through the artery. A flap 65 is shown at a high stress area 70 of the artery 5. Proximal to the stenosis 60 is an area of vulnerability 75, including a necrotic core 80. A rupture commonly occurs in an area such as the area of vulnerability 75.

FIGS. 5A and 5B illustratively depicts an imaging catheter 85 having a distal end 95 that is inserted into the stenotic area 20 of the artery 5. Alternatively, an imaging guidewire may be used to obtain intraluminal images. The imaging catheter 85 is inserted over a guidewire 90, which allows the imaging catheter 85 to be steered to the desired location in the artery 5. As depicted in FIGS. 5A and 5B, the imaging catheter 85 includes an imaging element 100 for imaging the diseased portions and normal portions of the artery 5. The imaging element 100 is, for example, a rotating ultrasound transducer, an array of ultrasound transducer elements such as phased array/cMUT, an optical coherence tomography element, infrared, near infrared, Raman spectroscopy, magnetic resonance (MRI), angioscopy or other type of imaging technology. Distal to the imaging element 100 is a tapered tip 105 which allows the imaging catheter 85 to easily track over the guidewire 90, especially in challenging tortuous, stenotic or occluded vessels. The imaging catheter 85 can be pulled back or inserted over a desired length of the vessel, obtaining imaging information along this desired length, and thereafter creating a volumetric model of the vessel wall, including the diseased and normal portions, from a set of circumferential cross-section images obtained from the imaging information. Some technologies, such as IVUS, allow for the imaging of flowing blood and thrombus.

FIGS. 6A-6C depict intraluminal imaging of a vessel 606 having a chronic total occlusion 604. Section 612 is a section of the vessel 606 proximal to the total chronic occlusion 604, which would receive enough contrast fluid to be discernible/present in an angiogram. Section 614 is a section of the vessel 606 that includes the total chronic occlusion 604 and portion of the vessel 606 that is distal to the total occlusion. Typically, section 612 would not receive enough contrast fluid to be discernible/present in an angiogram image.

As shown in FIGS. 6A-6C, a guidewire 610 is driven through a micro-channel or soft plaque within the total chronic occlusion 604, such that the guidewire 610 has passed the total chronic occlusion. The imaging catheter 608 with imaging element 602 is ridden over the guidewire 610 through the total occlusion 604. As an alternative to use of an imaging catheter, the guidewire itself may include an imaging element to obtain the intraluminal imaging data of the occluded vessel. The imaging catheter 608 includes an imaging element 602. Preferably, a radiopaque label 601 is co-located with the imaging element. The radiopaque label 601 allows the imaging element to be visualized and tracked while passing through section 614. The location of the imaging element 602 assists in the co-registration of the intraluminal imaging data with the extra-luminal imaging data. Particularly, tracking of the imaging element 602 allows one to align the cross-sectional imaging frames with the vessel present in the external imaging data and in a manner that more accurately represents the path of the occluded vessel.

In certain embodiments, the imaging catheter 608 obtains intraluminal imaging data (e.g. circumferential cross-sectional images) of the vessel prior to the total occlusion, within the total occlusion, and after the total occlusion. Using the obtained cross-sectional images, a two- or three-dimensional model of the vessel wall of sections 612, section 614, or both may be obtained. According to aspects of the invention, the two- or three-dimensional model of section 614 can be used to virtually reconstruct that section 614 of the vessel 606 that is missing/indiscernible in the angiogram.

In accordance with an aspect of an imaging system embodying the present invention, IVUS images are co-registered with the three-dimensional image depicted on the graphical display 160. Fiduciary points are selected when the imaging catheter is at one or more locations, and by combining this information with pullback speed information, a location vs. time (or circumferential cross-sectional image slice) path is determined for the imaging probe mounted upon the catheter. Co-registering cross-sectional intraluminal images (e.g. OCT or IVUS) with two-dimensional or three-dimensional images (e.g. angiogram or CT scan) from an external imaging modality allows for a two-dimensional or three-dimensional vasculature map. The cross-sectional IVUS or OCT imaging data can be used to fill in any data gaps present in an angiogram, thereby creating a more complete vasculature map/angiogram than previously possible.

Turning to FIG. 7, a set of steps are depicted for creating a vasculature map that includes one or more sections of a vessel not provided in an angiogram alone. During step 162, the imaging catheter is pulled back (or pushed forward) either manually or automatically through a blood vessel segment, and a sequence of circumferential cross-sectional IVUS (or OCT) image frames is acquired/created. During step 163 an angiographic image (or CT image) is formed of the blood vessel segment. The image is, for example, a two-dimensional image or, alternatively a three-dimensional image created from two or more angiographic views. During step 164, at least one fiduciary point is designated on the angiographic image, either by the user, or automatically by the imaging system. During step 166, the angiographic image and the information obtained from the imaging catheter during the pullback are aligned/correlated using the fiduciary point locating information. For step 168, a two- or three-dimensional vasculature map of the imaged vessel is displayed incorporating imaging data from both the IVUS imaging data and from the angiographic imaging data. The graphical representation of the imaged vessel is based at least in-part upon the angiographic imaging data and the IVUS imaging data in order to obtain a more complete vasculature map and more realistic representation of the vessel under examination.

Turning to FIG. 8, by combining or overlaying the three-dimensional map of imaging information over the three-dimensional image 160 of the vessel lumen, or over one or more two-dimensional views of the angiogram, a reconstruction 165 that more realistically represents the actual vessel is obtained, which is correct in its portrayal of vessel tortuosity, plaque composition and associated location and distribution in three dimensions. For example, a necrotic core which is located in the vessel in the sector between 30° to 90°, also having a certain amount of longitudinal depth, will appear on the reconstruction 165 with the same geometry. An augmented overall vessel diameter, due to thickened plaque, will also appear this way in the reconstruction 165. The additional information from the non-angiography imaging data makes displaying such vessel images possible. The steps of the procedure summarized in FIG. 7 facilitate co-registration of the IVUS information over a live two-dimensional angiographic image, giving the operator the ability to view a projection of the volume of plaque over a two-dimensional image of the lumen. The co-registered displayed graphical image allows an operator to make a more informed diagnosis, and also allows the operator to proceed with therapeutic intervention with the additional information provided by the co-registered displayed image guiding the intervention.

In the case of live two-dimensional or three-dimensional co-registration, one or more fiduciary points are selected first, followed by alignment by the system, and then simultaneous pullback and angiography or fluoroscopy. Note that in both co-registration in playback mode and co-registration in “live” mode, the information used by the system includes both the specific pullback speed being used (for example 0.5 millimeters per second) and the time vector of the individual image frames (for example IVUS image frames). This information tells the system where exactly the imaging element is located longitudinally when the image frame is (or was) acquired, and allows for the creation of an accurate longitudinal map.

Automatic fiduciary points are used, for example, and are automatically selected by the system in any one of multiple potential methods. A radiopaque marker on the catheter, approximating the location of the imaging element, for example is identified by the angiography system, creating the fiduciary point. Alternatively, the catheter has an electrode, which is identified by three orthogonal pairs of external sensors whose relative locations are known. By measuring field strength of an electrical field generated by the probe, the location of the electrode is “triangulated”.

FIG. 8 graphically depicts a reconstruction produced using the techniques discussed above. Three necrotic cores 80a, 80b and 80c have been identified. First necrotic core 80a is located at twelve o'clock circumferentially in the vessel and is identified as being located in the stenosis 60, and deep beneath a thickened cap. The location of the necrotic core 80a beneath the thickened cap suggests that this necrotic core is more stable than the other two necrotic cores—core 80b which is very close to the surface, and 80c which is also close to the surface. As shown in this reconstruction, and in relation to the first necrotic core 80a, the second necrotic core 80b is located at nine o'clock and the third necrotic core 80c is circumferentially located at four o'clock. This circumferential information is employed, for example, to localize application of appropriate treatment.

FIGS. 11-17 depict an exemplary technique for obtain accurate and axial circumferential co-registration of IVUS information (or other image information obtained via a probe inserted within a body) with a three-dimensional image 160 obtained from an external imaging modality data set. The following alignment steps may be used to align data present in both external imaging and intraluminal data set, or to align data present in a intraluminal imaging set with an external imaging data sets, in which data representing a certain segment of a vessel is absent from the external imaging data set.

Turning initially to FIG. 11 and FIG. 12, the illustrations are intended to represent the internal representation of information created/processed by the imaging/display system. However, in an illustrative embodiment, such information is presented as well as graphical displays rendered by the system, in the manner depicted in FIGS. 11 and 12 as a visual aid to users in a semi-automated environment. For example, a user can manually move the relative positioning of a sequence of IVUS frames with regard to linear displacement of a vessel as depicted in corresponding data values generated from an angiographic image.

Furthermore, as those skilled in the art will readily appreciate, the line graphs in FIGS. 11 and 12 corresponding to IVUS frames comprise a sequentially ordered set of discrete values corresponding to a sequence of “N” frames of interest. Similarly, values generated from angiographic image data are also taken at discrete points along a length of a vessel of interest. Thus, while depicted as continuous lines in the drawing figures, the values calculated from angiographic and IVUS information correspond to discrete points along the length of the vessel.

FIG. 11 includes a graph 320 depicting calculated/estimated lumen area as a function of IVUS image frame number for both angiography and IVUS. The graph depicted in FIG. 11 shows the effect of inaccurate co-registration between two imaging methods and associated measured parameters (e.g., lumen cross-section size). A line graph 330 representing lumen area calculated from IVUS information and a line graph 325 representing lumen area calculated from angiography information are shown in an exemplary case wherein the measurements are misaligned along a portion of a vessel.

FIG. 11 corresponds to a graphically displayed composite image depicted in FIG. 13 that includes a graphical representation of a three-dimensional angiographic image 335 and a graphical representation of corresponding IVUS information 340 where the two graphical representations are shifted by a distance (“D”) in a composite displayed image. The misalignment is especially evident because minimum luminal circumferential cross-section regions (i.e., the portion of the vessel having the smallest cross-section) in the images graphically rendered from each of the two data sets do not line up. The minimal lumen area calculated from the IVUS information at point 345 in FIG. 11 corresponds to the IVUS minimal lumen position 360 in FIG. 13. The lumen area calculated from the angiography information at point 350 in FIG. 11 corresponds to the angiography minimal lumen position 355 in FIG. 13. Note that in the illustrative example, thickness of the vessel wall is depicted as substantially uniform on IVUS. Thus, an IVUS image frame where the minimal lumen area occurs is also where the minimum vessel diameter exists.

A lumen border 380 is also shown in FIG. 13. In order to achieve axial alignment between the graphical representation of the three-dimensional angiographic image 335 and the graphical representation of corresponding IVUS information 340, an axial translation algorithm is obtained based upon a “best-fit” approach that minimizes the sum of the squared differences between luminal areas calculated using the angiographic and the IVUS image data.

The best axial fit for establishing co-registration between angiogram and IVUS data is obtained where the following function is a minimum.

$${\sum }_{n = 1}^{N} {( {A}_{Lumen} - {A}_{Angio} )}^{2}$$

with A_{Lumen}=IVUS lumen area for frames n=1, N and A_{Angio} angiography area for “frames” n=1, N (sections 1-N along the length of an angiographic image of a blood vessel). By modifying how particular portions of the angiographic image are selected, the best fit algorithm can perform both “skewing” (shifting all slices a same distance) and “warping” (modifying distances between adjacent samples).

Using the axial alignment of frames where the summation function is a minimum, a desired best fit is obtained. FIGS. 12 and 14 depict a result achieved by realignment of line graphs and corresponding graphical representations generated from the angiographic and IVUS data, depicted in a pre-aligned state in FIGS. 11 and 13, based upon application of a “best fit” operation on frames of IVUS image data and segments of a corresponding angiographic image,

FIG. 14 illustratively depicts a graphical representation of a three-dimensional lumen border 365 rendered from a sequence of IVUS image slices after axially aligning a three-dimensional angiographic data-based image with a graphical image generated from IVUS information for a particular image slice. The displayed graphical representation of a three dimensional image corresponds to the lumen border 380 shown in FIG. 15. The lumen border 380 is shown projected over a three-dimensional center line 385 obtained from the angiographic information. FIG. 15 also depicts a first angiography image plane 370 and a second angiography image plane 375 that are used to construct the three dimensional center line 385 and three-dimensional angiographic image 335. Such three-dimensional reconstruction is accomplished in any one of a variety of currently known methods. In order to optimize the circumferential orientation of each IVUS frame, an IVUS frame 400 depicting a luminal border is projected against the first angiography plane 370, where it is compared to a first two-dimensional angiographic projection 390. In addition, or alternatively, the IVUS frame 400 is projected against the second angiography image plane 375, where it is compared to the second two-dimensional angiographic projection 395 for fit. Such comparisons are carried out in any of a variety of ways including: human observation as well as automated methods for comparing lumen cross section images (e.g., maximizing overlap between IVUS and angiogram-based cross-sections of a vessel's lumen).

Positioning an IVUS frame on a proper segment of a graphical representation of a three-dimensional angiographic image also involves ensuring proper circumferential (rotational) alignment of IVUS slices and corresponding sections of an angiographic image. Turning to FIG. 16, after determining a best axial alignment between an IVUS image frame, such as frame 400, and a corresponding section of a three-dimensional angiographic image, the IVUS frame 400 is then rotated in the model by an angular displacement 405 (for example 1°) and the fit against the angiographic projections is recalculated. As mentioned above, either human or automated comparisons are potentially used to determine the angular displacement. After this has been done over a range of angular orientations, the best fit angular rotation is determined.

FIG. 17 depicts a graph 410 of best angle fit and frame number. During the pullback of the IVUS catheter, there may be some slight rotation of the catheter, in relation to the centerline of the blood vessel, and so, calculating the best angular fit for one IVUS frame does not necessarily calculate the best fit for all frames. The best angular fit is done for several or all frames in order to create the graph 410 including actual line 412 and fit line 414. The actual line 412 comprises a set of raw angular rotation values when comparing IVUS and angiographic circumferential cross-section images. The fit line 414 is rendered by applying a limit on the amount of angular rotation differences between adjacent frame slices (taking into consideration the physical constraints of the catheter upon which the IVUS imaging probe is mounted). By way of example, when generating the fit line 414, the amount of twisting between frames is constrained by fitting a spline or a cubic polynomial to the plot on the actual line 412 in graph 410.

For intraluminal imaging data representing a certain vessel segment not present or indiscernible in the external imaging data set, the intraluminal imaging data is used to fill in the missing data for the certain vessel segment. Preferably, the location of the intraluminal imaging data with respect to the certain vessel segment in the external imaging data set by tracking a radiopaque label co-located with an imaging element of the intraluminal imaging device. This allows the intraluminal cross-sectional image data frames to be stacked in sequential order corresponding to discrete points along the length of the vessel including within the occlusion and past the occlusion (which are not present in an external imaging data set). Alternatively or in addition to the radiopaque label, one or more distinct features common to the intraluminal and external imaging data sets may be used to align fill-in data representing a certain vessel segment missing from the external imaging data set with the intraluminal imaging data. In one embodiment, the one or more distinct features may be the luminal boundary of the vessel of the luminal area of the vessel. Luminal border detection techniques are described in U.S. Patent Publication No. 2008/0287795. In certain embodiments, the alignment of the certain vessel segment from the intraluminal imaging data with the external imaging data is enhanced by including in the alignment process intraluminal luminal imaging data that overlaps with the external imaging data. Incorporating a best-fit alignment of intraluminal image frames that correlate to a vessel present in the external imaging frames increases the likelihood that intraluminal image frames representing data not present or indiscernible from the external imaging frames are likewise aligned and are thus accurately representing the vessel in the vasculature. FIG. 9B shows a certain section 510 of a vessel 512, which was missing from an external image (shown in FIG. 9A), filled in with intraluminal imaging data representing section 510. FIG. 10B shows a certain section 504 of a vessel 512, which was missing from an external image (shown in FIG. 10A), filled in with intraluminal imaging data representing section 504.

In addition to the co-registration techniques described above, other techniques for co-registering intraluminal and external imaging data can be used in methods and systems of the invention. Co-registration of intraluminal and external imaging techniques suitable for use in system and methods of the invention are described in, for example, U.S. Patent Publication No. 2001/0319752, 2012/0004529, and 2010/0290693.

It is noted that although some techniques for co-using external imaging data and intraluminal imaging data are described hereinabove primarily with respect to external fluoroscopic/angiographic images and intraluminal IVUS images, the scope of the present invention includes applying the techniques described herein to other forms of external and intraluminal images and/or data, mutatis mutandis. For example, the external images may include images generated by fluoroscopy, CT, MRI, ultrasound, PET, SPECT, other extraluminal imaging techniques, or any combination thereof. Intraluminal images may include images generated by optical coherence tomography (OCT), near-infrared spectroscopy (NIRS), intravascular ultrasound (IVUS), endobronchial ultrasound (EBUS), magnetic resonance (MR), other endoluminal imaging techniques, or any combination thereof. Intraluminal data may include data related to pressure (e.g., fractional flow reserve), flow, temperature, electrical activity, or any combination thereof. Examples of the anatomical structure to which the aforementioned co-registration of external and intraluminal images may be applied include a coronary vessel, a coronary lesion, a vessel, a vascular lesion, a lumen, a luminal lesion, and/or a valve. It is noted that the scope of the present invention includes applying the techniques described herein to lumens of a subject's body other than blood vessels (for example, a lumen of the gastrointestinal or respiratory tract).

In advanced embodiments, a system 600, as shown in FIG. 18, may comprise an imaging engine 670 which has advanced image processing features, such as image tagging, that allow the system 600 to more efficiently process and display co-registered intravascular and external images. The imaging engine 670 may automatically highlight or otherwise denote areas of interest in the vasculature. The imaging engine 670 may also produce 3D renderings of the external images including at least some intravasculature data that is used to fill in missing data from the external images. In some embodiments, the imaging engine 670 may additionally include data acquisition functionalities (DAQ) 675, which allow the imaging engine 670 to receive the imaging data directly from the catheter 625 or collector 647 to be processed into images for display.

Other advanced embodiments use the I/O functionalities 662 of computer 660 to control the intravascular imaging 620 or external imaging 640. In these embodiments, computer 660 may cause the imaging assembly of catheter 625 to travel to a specific location, e.g., if the catheter 625 is a pull-back type. The computer 660 may also cause source 643 to irradiate the field to obtain a refreshed image of the vasculature, or to clear collector 647 of the most recent image. While not shown here, it is also possible that computer 660 may control a manipulator, e.g., a robotic manipulator, connected to catheter 625 to improve the placement of the catheter 625.

A system 700 of the invention may also be implemented across a number of independent platforms which communicate via a network 709, as shown in FIG. 19. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

As shown in FIG. 19, the intravascular imaging system 620 and the external imaging system 640 are key for obtaining the data, however the actual implementation of the steps, for example the steps of FIG. 19, can be performed by multiple processors working in communication via the network 709, for example a local area network, a wireless network, or the internet. The components of system 700 may also be physically separated. For example, terminal 767 and display 780 may not be geographically located with the intravascular imaging system 620 and the external imaging system 640.

As shown in FIG. 19, imaging engine 859 communicates with host workstation 433 as well as optionally server (not shown) over network 409. In some embodiments, an operator uses host workstation 733, computer 660, or terminal 767 to control system 700 or to receive images. An image may be displayed using an I/O 662, 737, or 771, which may include a monitor. Any I/O may include a monitor, keyboard, mouse or touch screen to communicate with any of processor 665, 741, or 775, for example, to cause data to be stored in any tangible, nontransitory memory 367, 745, or 779. Server generally includes an interface module to communicate over network 409 or write data to data file. Input from a user is received by a processor in an electronic device such as, for example, host workstation 733, terminal 767, or computer 660. In certain embodiments, host workstation 733 and imaging engine 855 are included in a bedside console unit to operate system 700.

In some embodiments, the system may render three dimensional imaging of the vasculature or the intravascular images. An electronic apparatus within the system (e.g., PC, dedicated hardware, or firmware) such as the host workstation 733 stores the three dimensional image in a tangible, non-transitory memory and renders an image of the 3D tissues on the display 780. In some embodiments, the 3D images will be coded for faster viewing. In certain embodiments, systems of the invention render a GUI with elements or controls to allow an operator to interact with three dimensional data set as a three dimensional view. For example, an operator may cause a video affect to be viewed in, for example, a tomographic view, creating a visual effect of travelling through a lumen of vessel (i.e., a dynamic progress view). In other embodiments an operator may select points from within one of the images or the three dimensional data set by choosing start and stop points while a dynamic progress view is displayed in display. In other embodiments, a user may cause an imaging catheter to be relocated to a new position in the body by interacting with the image.

In some embodiments, a user interacts with a visual interface and puts in parameters or makes a selection. Input from a user (e.g., parameters or a selection) are received by a processor in an electronic device such as, for example, host workstation 733, terminal 767, or computer 660. The selection can be rendered into a visible display. In some embodiments, an operator uses host workstation 733, computer 660, or terminal 767 to control system 700 or to receive images. An image may be displayed using an I/O 762, 737, or 771, which may include a monitor. Any I/O may include a keyboard, mouse or touch screen to communicate with any of processor 665, 741, or 775, for example, to cause data to be stored in any tangible, nontransitory memory 667, 745, or 779. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections). In certain embodiments, host workstation 733 and imaging engine 855 are included in a bedside console unit to operate system 700.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, NAND-based flash memory, solid state drive (SSD), and other flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell networks (3G, 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include programming language known in the art, including, without limitation, C, C++, Perl, Java, ActiveX, HTML5, Visual Basic, or JavaScript.

A computer program does not necessarily correspond to a file. A program can be stored in a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment) into patterns of magnetization by read/write heads, the patterns then representing new collocations of information desired by, and useful to, the user. In some embodiments, writing involves a physical transformation of material in tangible, non-transitory computer readable media with certain properties so that optical read/write devices can then read the new and useful collocation of information (e.g., burning a CD-ROM). In some embodiments, writing a file includes using flash memory such as NAND flash memory and storing information in an array of memory cells include floating-gate transistors. Methods of writing a file are well-known in the art and, for example, can be invoked automatically by a program or by a save command from software or a write command from a programming language.

In certain embodiments, display 780 is rendered within a computer operating system environment, such as Windows, Mac OS, or Linux or within a display or GUI of a specialized system. Display 780 can include any standard controls associated with a display (e.g., within a windowing environment) including minimize and close buttons, scroll bars, menus, and window resizing controls. Elements of display 780 can be provided by an operating system, windows environment, application programming interface (API), web browser, program, or combination thereof (for example, in some embodiments a computer includes an operating system in which an independent program such as a web browser runs and the independent program supplies one or more of an API to render elements of a GUI). Display 780 can further include any controls or information related to viewing images (e.g., zoom, color controls, brightness/contrast) or handling files comprising three-dimensional image data (e.g., open, save, close, select, cut, delete, etc.). Further, display 780 can include controls (e.g., buttons, sliders, tabs, switches) related to operating a three dimensional image capture system (e.g., go, stop, pause, power up, power down).

In certain embodiments, display 780 includes controls related to three dimensional imaging systems that are operable with different imaging modalities. For example, display 780 may include start, stop, zoom, save, etc., buttons, and be rendered by a computer program that interoperates with IVUS, OCT, or angiogram modalities. Thus display 780 can display an image derived from a three-dimensional data set with or without regard to the imaging mode of the system.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for imaging a vessel, the method comprising
obtaining external imaging data of a vessel, wherein data representing a specific portion of the vessel is absent from the external imaging data;
obtaining intraluminal imaging data of the specific portion of the vessel; and
co-registering the external imaging data with the intraluminal imaging data to construct an external image of the vessel that includes the specific portion of the vessel.

2. The method of claim 1, wherein the specific portion of the vessel comprises a total chronic occlusion.

3. The method of claim 1, wherein the external imaging data comprises angiographic image data.

4. The method of claim 1, wherein the external image is three-dimensional.

5. The method of claim 1, wherein the external image is two-dimensional.

6. The method of claim 1, wherein the intraluminal imaging data is obtained using optical coherence tomography, ultrasound technology, intravascular spectroscopy, or photo-acoustic tomography.

7. The method of claim 2, further comprising
inserting an intraluminal device comprising an imaging element into the chronic total occlusion; and
imaging the total chronic occlusion to obtain the intraluminal imaging data.

8. A method for imaging a vessel, the method comprising
obtaining angiographic imaging data of a vessel, wherein data representing a chronic total occlusion in the vessel is absent from the angiographic imaging data;
obtaining intraluminal imaging data of the chronic total occlusion; and
co-registering the angiographic imaging data with the intraluminal imaging data to construct an angiographic image of the vessel that includes the chronic total occlusion.

9. The method of claim 8, wherein the external image is three-dimensional.

10. The method of claim 8, wherein the external image is two-dimensional.

11. The method of claim 8, wherein the obtaining intraluminal imaging data step comprises:
inserting an intraluminal device comprising an imaging element into the chronic total occlusion; and
imaging the total chronic occlusion to obtain the intraluminal imaging data.

12. A system for imaging a vessel, comprising:
a central processing unit (CPU); and
a storage device coupled to the CPU and having stored there information for configuring the CPU to:
acquire external imaging data of a vessel, wherein data representing a specific portion of the vessel is absent from the external imaging data;
acquire intraluminal imaging data of the specific portion of the vessel; and
co-register the external imaging data with the intraluminal imaging data to construct an external image of the vessel that includes the specific portion of the vessel.
